(19) United States
(12) Patent Application Publication
(10) Pub. No.: US 2026/0009811 A1
(43) Pub. Date: Jan. 8, 2026

(54) NANOROBOT FOR RECOGNITION OF PROTEINS IN MEDIUMS AT ULTRA-LOW CONCENTRATION

(71) Applicant: The University of Hong Kong, Hong Kong (CN)

(72) Inventors: Ning XI, Hong Kong (CN); Yuxuan XUE, Hong Kong (CN); William Shu Biu YEUNG, Hong Kong (CN); Yin Lau LEE, Hong Kong (CN)

(21) Appl. No.: 19/253,379

(22) Filed: Jun. 27, 2025

Related U.S. Application Data
(60) Provisional application No. 63/667,315, filed on Jul. 3, 2024.

(51) Int. Cl. G01N 35/00; G01N 33/543; G01N 33/76; G16B 40/10

(52) U.S. Cl. CPC G01N 35/0099; G01N 33/54366; G01N 33/76; G01N 35/00722; G16B 40/10; G01N 2333/59

(57) ABSTRACT

Disclosed herein is a system for the quantification of low-concentration analytes in a sample, such as culture of human embryos. The system allows for the accurate identification and quantification of an analyte, for example a protein, at a resolution and sensitivity less than 1 pg/ml. The disclosed system provides a non-invasive, rapid, and precise measurement platform, thereby enabling better patient care through more informed medical decision-making. Also disclosed herein is a method for using the disclosed system for quantifying analytes in a sample.

### CROSS-REFERENCE TO RELATED APPLICATIONS

This application claims the benefit of and priority to U.S. Provisional Application No. 63/667,315 filed Jul. 3, 2024, which is herein incorporated by reference in its entirety.

### FIELD OF THE INVENTION

The field of the invention is generally in the field of biomedical diagnostics, specifically quantification of proteins.

### BACKGROUND OF THE INVENTION

There is a need for more precise and reliable methods of quantifying essential protein concentration of human chorionic gonadotropin (hCG) in embryo spent medium, which is an important biomarker for pregnancy and certain medical conditions, including trophoblastic diseases and some cancers. Traditional methods for protein detection, such as enzyme-linked immunosorbent assay (ELISA) and radioimmunoassay (RIA), often suffer from limitations in sensitivity, specificity, and the need for extensive sample preparation. These conventional techniques may also experience cross-reactivity with other substances in the sample, leading to false positives or negatives, which can significantly impact patient diagnosis and treatment. The system described herein overcomes these challenges by allowing for direct, label-free detection of hCG molecules with higher accuracy and resolution. The improved robotic technique minimizes the risks of cross-reactivity and false readings, providing a robust and dependable tool for clinicians and researchers. Consequently, the system fulfills a long-felt need in the medical diagnostic field for a method that ensures swift, non-invasive for cultured embryo scenarios, and precise measurement of proteins, thereby enhancing the standard of care and potentially improving clinical outcomes.

### SUMMARY OF THE INVENTION

Disclosed herein is a system to for the detection of analytes at low concentrations in a sample. The disclosed system involves a measurement module, a signal communication module, precise sample-tip positioning, and an AI-based data processing software, with integration of real-time image updates, sample-tip positioning accuracy, and the augmented reality system for an enhanced user interface in protein recognition measurements.

Also disclosed herein is a method for detecting concentration levels of an analyte using the disclosed system, the method involving (i) functionalizing the probe and platform, (ii) calibrating and setting up the system, (iii) carrying out the protein measurement experimentation; and (iv) analyzing the acquired data using an AI-based data analytic system.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations. It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed method and compositions and together with the description, serve to explain the principles of the disclosed method and compositions.

FIG. 1A is a schematic showing the architecture and hardware design of the nanorobotic system for low-concentration protein detection. The system's main body consists of a measurement module and a signal communication module. The motion of the probe platform is controlled by an XYZ motion platform driven by motors for large-distance movement. For precise motion control, a 3-dimensional piezoelectric element is attached to the platform. FIG. 1B is a schematic that illustrates the fabricated scaffold disk with different types of protein bindings, securely mounted on the XY piezo motion unit for accurate measurements. The probe's motion is controlled by the Z-axis piezoelectric actuator, while the feedback loop of the probe is completed by a PVDF thin film attached to the probe's cantilever.

FIG. 2 is a schematic showing the measurement principle of the multi-probe nanorobotic system during the interaction between the functionalized probes and the captured proteins. The scaffold disk captures various types of proteins based on the first antibody attached to its surface. Each probe-PVDF end-effector, functionalized with a specific secondary antibody, binds exclusively to its corresponding target protein. The rupture event is detected by the probe, converting the biochemical signal into a measurable force signal.

FIGS. 3A and 3B show the fabricated probe with focus ion beam.

FIG. 3A shows the focus ion beam fabricate of the probe tip within the scan of scanning electron microscopy. FIG. 3B shows the fabricated high aspect ratio probe.

FIG. 4 shows the schematic for the fabrication of the scaffold disk and functionalized probe to perform the protein measurement. The scaffold disk is following the protocol of binding targeted protein antibody on the mica with the coated layer MPTMS; GMBS; G-protein. The secondary antibody is linked to the probe with the PEG linker.

FIG. 5 shows the principle of the rupture events recording for the antigen-antibody as the force readings and the construction of force images. The left graph shows the spatial distribution of the protein on the scaffold disk; the right graph shows the force-distance curve for the detection of rupture event.

FIG. 6 shows the high-resolution topography of the HCG captured disk with different protein concentration and height profile of HCG-HCG antibody.

FIG. 7 shows the rupture force readings distribution histogram with the different concentration (0-200 pg/ml).

FIG. 8 shows the regression curve of rupture force to protein concentration. The range of the regression curve is 0-50 pg/ml.

FIG. 9 shows imaging standard assay of the HCG with concentration.

The graph shows the typical protein detection images with the HCG concentration of 0; 1; 5; 10; 50; 100 pg/ml.

FIG. 10 shows the architectural of convolutional neural network (CNN) based deep learning approach for the acquisition of protein concentration from force images.

FIG. 11 shows the architecture of CNN-regression network.

FIG. 12 shows the training results of repression curve. The test loss will reach to less than 50.

FIG. 13 shows the protein readings for the HCG from human spent culture medium for the embryo using the inventor.

FIG. 14 shows the force images and distribution of the successful and unsuccessful pregnancy spent culture medium to the targeted protein.

FIG. 15 shows the protein concentration histogram for the successful and unsuccessful pregnancy spent culture medium to HCG.

### DETAILED DESCRIPTION OF THE INVENTION

### I. Definitions

“Nanorobotic system” refers to the design, engineering, and use of extremely small robotic devices—typically measured in nanometers (1 nanometer=10⁻⁹ meters)—that can perform specific tasks at the molecular or cellular level. Examples of components that can be included in nanorobotic systems include: sub-nanoscale motion control unit, force-sensing feedback, sensors, actuators, power source, control unit, and/or communication system. The motion-control unit combines a high-performance piezoelectric (PZT) actuator with an optimized structural design, while an embedded real-time nonlinear control algorithm preserves accuracy at high speeds. Force-sensing feedback is obtained from a piezo electric film (e.g., a piezo electric thin film such as a PVDF thin film) and its integrated circuitry deposited on an end-effector, together with pre-calibrated three-dimensional deformation-to-force models.

“Operably linked” refers to the connection of at least two components in a system allowing them to work together via technology including, but not limited to, integrated circuits, electrical cables, ethernet, internet, intranet, Bluetooth, near field communication, WiFi, or a combination thereof. In one exemplary form, “operably linked” refers to a functional interaction between two or more components in a system, allowing them to work together to achieve a shared objective.

The term “real-time” refers to data transmission to a user interface of a computer-implemented method, system, tool, or device within 1, 2, 3, 4, 5, 10, 15, 20, or no more than 30 minutes after the computer-implemented method, system/tool/device is used to detect a concentration of an analyte in a sample. The transmitted data can be the concentration of the analyte in a sample based, in part, on processing signals generated by the computer-implemented method, system, tool, or device.

### II. Compositions

Disclosed herein is a nanorobotic system for the quantification of a low-concentration analyte, e.g. protein, in a sample. In some embodiments, the sample is a spent medium, for example a culture of human embryos. Also disclosed herein is a method of using the nanorobotic system for the detection and measurement of an analyte, e.g. protein. In some embodiments, the analyte is a biomarker critical for pregnancy testing and monitoring, as well as certain cancers. The disclosed system contains a nanorobotic system, chip manufacturing protocol, and/or an artificial intelligent (AI) processing software that allows for the accurate identification and quantification of the analyte, e.g. protein, with high resolution and sensitivity (less than 1 pg/mL). Preferably, the chip manufacturing protocol is consistent with that of the platform manufacturing protocol (e.g., scaffold disk protocol). The disclosed nanorobotic system contains a measurement module, a signal communication module, and precise sample-tip positioning capabilities. The disclosed nanorobotic system provides a significant improvement over existing diagnostic methods by offering a non-invasive, rapid, and highly precise measurement platform.

In some embodiments, the system quantifies analyte concentrations between 0.1 pg/ml and 100 pg/ml, between 0.1 pg/ml and 90 pg/ml, between 0.1 pg/ml and 80 pg/ml, between 0.1 pg/ml and 70 pg/ml, between 0.1 pg/ml and 60 pg/ml, between 0.1 and 50 pg/ml, between 0.1 pg/ml, and 40 pg/ml, between 0.1 pg/ml and 30 pg/ml, between 0.1 pg/ml and 20 pg/ml, between 0.1 pg/ml and 10 pg/ml, or between 0.1 pg/ml and 1 pg/ml. Preferably, the system quantifies analyte concentrations less than 1 pg/ml.

The disclosed system quantifies analyte, e.g., protein concentrations, in a sample. In some embodiments, the sample is an embryo spent medium. In some embodiments, the analyte is a protein. In some embodiments, the protein is a Human chorionic gonadotropin (hCG), which is a biomarker for pregnancy and certain medical conditions, including trophoblastic diseases and some cancers. The disclosed system is a nanorobotic system that allows for direct, label-free detection of an analyte, e.g. protein, with high accuracy and resolution. The disclosed system minimizes the risks of cross-reactivity and false readings, providing a robust and dependable tool for clinicians and researchers.

The disclosed nanorobotic system incorporates a measurement module, a signal communication module, precise sample-tip positioning and an AI-based data processing software.

The disclosed system and methods can be understood more readily by reference to the following detailed description of particular embodiments and the Example included therein and to the Figures and their previous and following description.

### A. Measurement Module

The measurement module of the disclosed system includes a configuration for surface topography assessment of a molecule and a platform (e.g. scaffold disk) to immobilize an analyte for concentration quantification. In some embodiments, the configuration uses a probe and cantilever utilized in Atomic Force Microscopy (AFM), lasers utilized in optical tweezers, or magnets utilized in magnetic tweezers.

The disclosed system incorporates a scaffold platform to immobilize the analyte, e.g. protein, to be quantified and a measurement protocol. Executing analyte recognition measurement involves four main steps: functionalizing the probe and platform, calibrating and setting up the nanorobotic system, carrying out the experimentation, and data analysis.

### 1. Cantilever and Probe

In some embodiments, the measurement module of the disclosed system includes a probe. The probe of the measurement module is designed to manipulate mechanical traits of an individual molecule and determine rupture forces applied at specific locations with high spatial resolution. In some embodiments, the probe is constructed from a commercially available cantilever, for example an aqueous cantilever and modified to have a suitable tip radius and/or inner tip distance.

In some embodiments, the probe has a tip radius between 10 nm and 100 nm, between 10 nm and 90 nm, between 10 nm and 80 nm, between 10 nm and 70 nm, between 10 nm and 60 nm, between 20 nm and 60 nm between 30 nm and 60 nm, or between 40 nm and 60 nm.

In some embodiments, the probe has an inter-tip distance between 100 nm and 300 nm, between 120 nm and 300 nm, between 140 nm and 300 nm, between 160 nm and 300 nm, between 180 nm and 300 nm, between 180 nm and 280 nm, between 180 nm and 260 nm, between 180 nm and 240 nm, or between 180 nm and 220 nm.

In some embodiments, the probe has a length between 50 μm-300 μm, 50 μm-250 μm, 50 μm-200 μm, 50 μm-150 μm, 100 μm-150 μm, 100 μm-200 μm, 100 μm-250 μm, or 100 μm-300 μm.

The probe is functionalized by modifying the probe's surface or structure with molecules and/or chemical groups that bind to an analyte, for example the analyte is a protein, immobilized on the platform. In some embodiments, the molecule, or chemical group used to functionalize the probe is a secondary antibody, for example a human chorionic gonadotropin (HCG) antibody.

In some embodiments, the probe undergoes silanization to promote functionalization of the probe by attaching alkoxysilane molecules to surface of the probe. In some embodiments, alkoxysilane molecules are attached to the surface of the probe with a suitable reagent for silanization, for example (3-Aminopropyl)triethoxysilane (APTES), (3-Mercaptopropyl)trimethoxysilane (MPTMS), (3-Glycidyloxypropyl)trimethoxysilane (GPTMS), (3-Chloropropyl)triethoxysilane (CPTES), (3-Methacryloxypropyl) trimethoxysilane (MAPTMS), (3-Triethoxysilylpropyl)succinic anhydride (TESPSA), or a combination thereof. Preferably, the reagent for silanization of the probe is APTES.

In some embodiments, a linker is used to attach the probe with the molecules and/or chemical groups that binds to an analyte. In some embodiments, the linker is a polyethylene glycol (PEG) linker, alkyl chain, disulfide bond, amide linker, ester linker, maleimide linker, hydrazone linker, thioether linker, carbamate linker, or a linker derived from click chemistry, for example triazoles. In some embodiments, the linker is constructed from a cross-linking reagent. In some embodiments, the cross-linking reagent is glutaraldehyde, formaldehyde, disuccinimidyl suberate (DSS), bis(sulfosuccinimidyl)suberate (BS3), ethylene glycol bis(succinimidyl succinate) (EGS), dithiobis(succinimidyl propionate) (DSP), 1-ethyl-3-(3-dimethylaminopropyl)carbodiimide (EDC), N-hydroxysuccinimide (NHS), O,O′-Bis[2-(N-Succinimidyl-succinylamino) ethyl]polyethylene glycol, or a combination thereof. Preferably, the probe is functionalized with APTES attached to a PEG linker, which is further attached to an HCG secondary antibody.

The probe and cantilever can be constructed from a material suitable for AFM applications. For example, the probe and cantilever are constructed from silicon, silicon nitride, doped silicon, silicon oxide, aluminum, or polymers, for example Poly(methyl methacrylate) (PMMA). In some embodiments, one or more probes and cantilevers are attached to each other via a probe platform. In some embodiments, the motion of the probe platform is managed by an XYZ motion platform for large-distance movements. In some embodiments, precise motion control of the probe platform is managed by a 3-dimensional piezoelectric element.

### 2. Platform (e.g., Scaffold Disk)

In some embodiments, the measurement module of the disclosed system includes a platform (e.g., scaffold disk) to immobilize the analyte for quantification. In some embodiments, the scaffold disk has one or more layers of material, preferably 5 layers of material.

In some embodiments, the platform (e.g., scaffold disk) contains a glass layer or a mica material layer, for example the mica material is muscovite mica, e.g., A1 grade muscovite mica. In some embodiments, the mica material layer is coated with alkoxysilane molecules attached to the surface of the mica material layer with a suitable reagent for silanization. In some embodiments, the reagent for silanization of the mica material layer is (3-aminopropyl)triethoxysilane (APTES), (3-mercaptopropyl)trimethoxysilane (MPTMS), (3-glycidyloxypropyl)trimethoxysilane (GPTMS), (3-chloropropyl)triethoxysilane (CPTES), (3-methacryloxypropyl) trimethoxysilane (MAPTMS), (3-triethoxysilylpropyl)succinic anhydride (TESPSA), trimethoxypropylsilane (TMPS), octadecyltrichlorosilane (OTS), hexamethyldisilazane (HMDS), tetraethoxysilane (TEOS), or a combination thereof. Preferably, the reagent for silanization of the mica material layer is MPTMS.

In some embodiments, the scaffold platform further contains a protein crosslinker layer. The protein crosslinker layer is formed from any suitable protein crosslinker reagent. Exemplary embodiments of protein crosslinker reagent include but are not limited to amine-anime crosslinker, amine to carboxyl crosslinker, thiol crosslinker, heterobifunctional cross linker, or a amine-to-sulfhydryl crosslinker. In some embodiments, the protein crosslinker reagent is an amine-to-sulfhydryl crosslinker, such as N-γ-maleimidobutyryl-oxysuccinimide ester (GMBS).

In some embodiments, the scaffold platform further contains a G-protein layer, containing protein G. This layer facilitates the binding of the scaffold layer. The scaffold layer selectively captures and immobilizes the analyte to be quantified. In some embodiments, the scaffold layer is an antibody scaffolding layer for capturing analytes, for example anti-HCG antibodies (hCGab). In some embodiments, the scaffold layer is made of a first HCG antibody (e.g., first hCGab).

Once the platform is equipped with a scaffold layer, the analyte attaches to the surface of the scaffold layer to form a complex on the surface of the platform. In some embodiments, the analyte is in a sample, for example a target protein medium or embryo medium. In some embodiments, the complex formed on the surface of the platform is a hCG-hCGab complex.

### B. Signal Communication Module

The signal communication module of the disclosed system includes piezoelectric materials, circuits, signal detector, and an augmented reality system.

### 1. Piezoelectric Material

The signal communication module of the disclosed system includes piezoelectric materials to convert mechanical stress into measurable electrical signals. In some embodiments, piezoelectric materials are configured in a 3-dimensional piezoelectric element separated into X piezoelectric unit positioned along the X-axis of the platform, Y piezoelectric unit positioned along the Y-axis of the platform, and Z piezoelectric unit positioned on the probe and cantilever. In some embodiments, the XYZ piezoelectric units also function as XYZ piezoelectric scanners, with all motions controlled by a main computer either automatically and/or controlled by a user.

The piezoelectric materials can be any suitable piezoelectric material, for example a quartz, a lead zirconate a titanate, a barium titanate, a zinc oxide, an aluminum nitride, a polyvinylidene fluoride (PVDF), a lithium niobate, a gallium orthophosphate, or a tourmaline. Preferably, the piezoelectric material contains a PVDF.

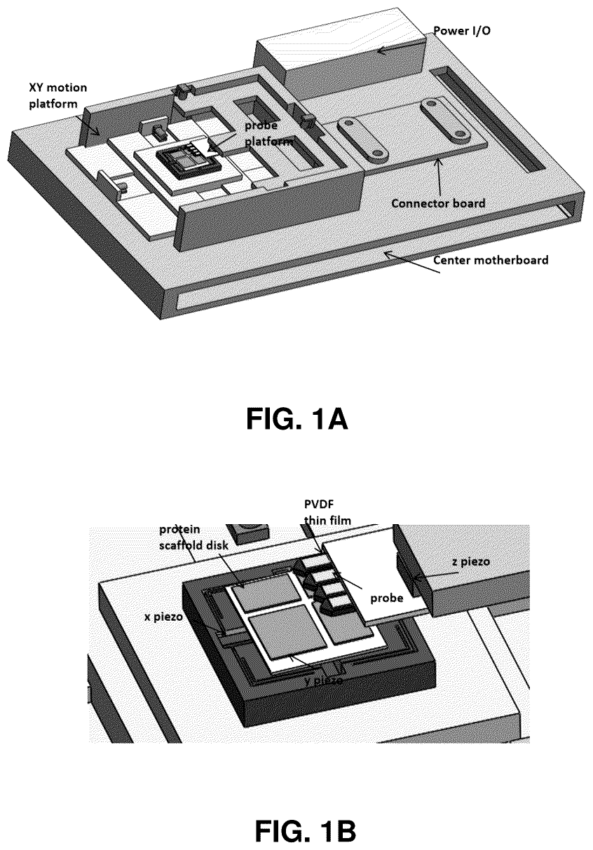

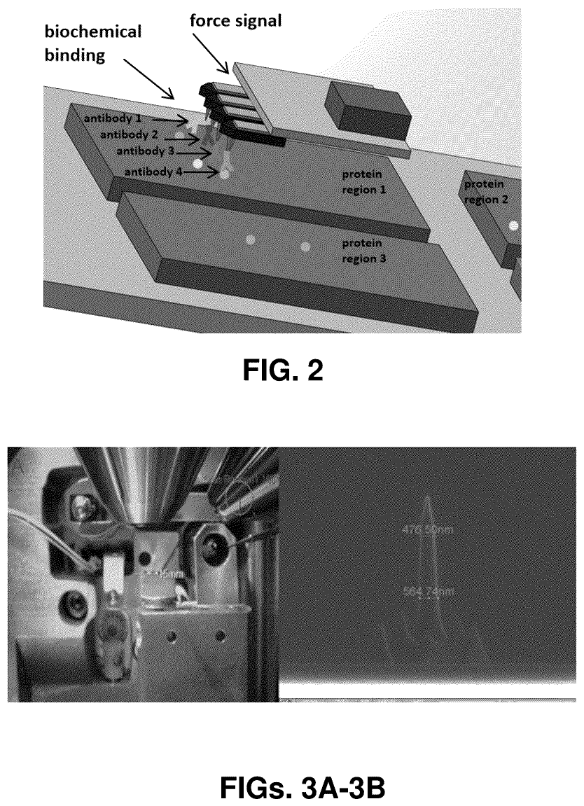

A nanorobotic system, as described in FIG. 1A, is designed for low-concentration analyte, e.g. protein, detection. The scaffold platform, shown in FIG. 1B, is securely mounted on the X and Y piezo motion unit and features various types of protein bindings. The Z-axis piezoelectric actuator controls the probe's motion, while a PVDF thin film attached to the probe's cantilever completes the feedback loop. The XYZ piezoelectric units also function as scanners, with all motions controlled by a main computer.

The analytes are captured on the scaffold platform through the first antibodies attached to its surface. Each probe is functionalized with a specific secondary antibody, allowing it to bind exclusively to a corresponding target protein. The rupture event for each probe is detected individually, converting the biochemical signals into measurable force signals. These force signals are transmitted via the piezoelectric materials as voltage outputs to the real-time control module, which is connected to a signal connection board for synchronized data collection and signal output. This multi-probe setup enables the simultaneous recognition of different analytes (e.g., proteins) with high specificity and efficiency.

### 2. Circuits

The signal communication module of the present application includes a connector board and center mother board. In some embodiments, the connector board acts as an interface, facilitating communication between PZT materials, sensors, motors and center motherboard disclosed herein. In some embodiments, a center mother board processes a control feedback command and analyzes a data.

### C. Precise Sample-Tip Positioning

The precise sample-tip positioning of the disclosed system includes compensating for the motion of nanoscale entities, the probe tip, and the microenvironment of the nanoscale entities. Precise sample-tip positioning can be achieved through rapid scanning during operation, with the nanorobotic system continuously recalibrating its position using environmental landmarks.

In some embodiments, the precise sample tip positioning is achieved by establishing the initial background for nanoscale imaging in the environment as the baseline and continuously updating locally based on real-time measurement. In some embodiments, the system's local scan feature utilizes adaptive scanning patterns to detect the new position of the analyte, e.g. protein. The scanning patterns of the adaptive scanning patterns are changed according to the observed object, with dense scanning points with the region of interest, and sparse scanning in the other region. This adjustment is possible by accurately recovering the analyte's actual position through the measurement of height profiles as determined by the cantilever and functionalized probe. In some embodiments, this method enables the rapid recovery of the true position of the analyte, e.g. protein, in less than 10 milliseconds, 20 milliseconds, 30 milliseconds, 40 milliseconds, 50 milliseconds or 60 milliseconds.

### D. AI-Based Analytic System

The AI-based analytic system of the disclosed system involves a computer-implemented system configured to analyze the surface topography data of the analyte and quantify the analyte concentration. In some embodiments, the AI-based analytic system is based on deep learning, symbolic AI, rule-based systems, classical machine learning, evolutionary algorithms, swarm intelligence, reinforcement learning, fuzzy logic systems, case-based reasoning, Bayesian networks, constraint satisfaction problems, or a combination thereof. In some embodiments, the AI-based analytic system is based on deep learning, for example neural networks, such as convolutional neural networks. Examples of neural networks suitable for image analysis include, but are not limited to, convolutional neural networks, AlexNet, VGGNet, ResNet, Inception Network, EfficientNet, U-net, Mask R-CNN, Vision Transformer, etc.

In some embodiments, the AI-based analytic system is configured to visualize the height and rupture force of the analyte complexes immobilized on the substrate using atomic force microscopy (AFM) and processing the data to plot the relative distributions of visualized objects by height and adhesive force.

In some embodiments, the signal processing is handled by two integrated computers: a conventional interface computer dedicated to post-processing tasks, and an augmented interface computer responsible for real-time control, enabling both automated operation and human interaction through a haptic device.

### III. Methods of Using

Methods of using the disclosed system for quantifying an analyte, e.g. protein, in a sample are also described herein. An exemplary protocol of using the disclosed nanorobotic system for the accurate measurement of HCG concentrations, detected through binding events of HCG-HCG antibody complexes, are as follows: a cleaved mica substrate is coated with protein G to facilitate the binding of the HCG antibody scaffold layer. This layer is capable of selectively capturing HCG proteins. Once the substrate is equipped with a dense antibody layer, the target protein medium or embryo medium are incubated on the scaffold platform to form the HCG-HCGab complex, and any unbound components are subsequently washed away. The second step involved fabricating a functionalized probe using a similar protocol, with the addition of a secondary HCG antibody. The probes were then blocked with a BSA medium to prevent non-specific binding via the PEG linker.

In the third step, the nanorobotic system was employed to visualize the height and rupture force of the protein complexes immobilized on the substrate, allowing for the determination of specific rupture forces applied to the functionalized probe at each location with high spatial resolution.

The final step involved processing the measurement data, where the relative distributions of visualized objects by height and adhesive force, r(h), f(h), were plotted, and the number of these objects per unit area was calculated.

The system utilizes a semantic compressive feedback control mechanism, which streamlines the recognition process by forgoing traditional feature extraction and complex calibrations, thereby reducing computational load and accelerating image processing. In some embodiments, the compressed sensing is the learning-based framework that modifies a sampling matrix (W), basis matrix (D), and projection matrix (Q) to reduce scanning points while ensuring high-quality reconstruction, improving sampling and reconstruction.

The disclosed systems and methods can be further understood through the following numbered paragraphs.

1. A nanorobotic system for detecting a concentration of an analyte in a sample, the system containing:
a measurement module containing a functionalized probe and a platform (e.g. a scaffold disk),
   a signal communication module containing a material capable of generating and/or transmitting one or more signals optionally selected from electric signals, electrochemical signals, optical signals, mechanical signals (e.g., originating from pressure, tensile/compressive forces, accelerations, vibrations, strain/deformation, displacement, and/or shearing), acoustic signals, temperature signals, digital signals, and
   a sample-tip positioning module (e.g., precise sample tip position module),
   wherein the system is operably linked to an artificial intelligence (AI)-based data analytic system configured to interpret data containing analyte-functionalized probe interaction images and/or convert the data into a concentration reading,
   optionally wherein the AI-based data analytic system utilizes a machine learning AI platform, deep learning AI platform (e.g., neural networks such as convolutional neural networks (CNNs)), or a combination thereof, for interpreting the data (e.g., analyte-functionalized probe interaction images, such as protein-protein interaction images) and/or converting the data into concentration readings.
      2. The nanorobotic system of paragraph 1, wherein the nanorobotic system is configured to implement a semantic compressive feedback control mechanism to interpret the data optionally by streamlining an image recognition process and/or reducing computational load.
      3. The nanorobotic system of paragraph 1 or 2, wherein the functionalized probe contains metals, metal alloys, carbon-based materials, polymers, magnetic materials, semiconductor materials, nucleic acid origamis (e.g., DNA origamis), or combinations thereof.
      4. The nanorobotic system of any one of paragraphs 1 to 3, wherein the functionalized probe contains alkoxysilane molecules on an external surface, wherein the alkoxysilane molecules are (3-aminopropyl)triethoxysilane (APTES), (3-mercaptopropyl)trimethoxysilane (MPTMS), (3-glycidyloxypropyl)trimethoxysilane (GPTMS), (3-chloropropyl)triethoxysilane (CPTES), (3-methacryloxypropyl) trimethoxysilane (MAPTMS), (3-triethoxysilylpropyl)succinic anhydride (TESPSA), or a combination thereof.
      5. The nanorobotic system of any one of paragraphs 1 to 4, wherein the functionalized probe contains a linker, wherein the linker contains a polyethylene glycol (PEG) linker, alkyl chain, disulfide bond, amide linker, ester linker, maleimide linker, hydrazone linker, thioether linker, carbamate linker, or a linker derived from click chemistry (e.g., triazole).
      6. The nanorobotic system of any one of paragraphs 1-5, wherein the functionalized probe contains molecules and/or chemical groups that bind to the analyte immobilized on the platform.
      7. The nanorobotic system of any one of paragraphs 1-6, wherein the platform (e.g., scaffold disk) contains a mica material (e.g., a mica material layer).
      8. The nanorobotic system of any one of paragraphs 1-7, wherein the functionalized probe has:
(i) a tip radius between 10 nm and 100 nm, between 10 nm and 90 nm, between 10 nm and 80 nm, between 10 nm and 70 nm, between 10 nm and 60 nm, between 20 nm and 60 nm between 30 nm and 60 nm, or between 40 nm and 60 nm, and/or
(ii) a length between 50-300 μm, 50-250 μm, 50-200 μm, 50-150 μm, 100-150 μm, 100-200 μm, 100-250 μm, or 100-300 μm.
   9. The nanorobotic system of any one of paragraphs 1-8, wherein the functionalized probe has an inter-tip distance between 100 nm and 300 nm, between 120 nm and 300 nm, between 140 nm and 300 nm, between 160 nm and 300 nm, between 180 nm and 300 nm, between 180 nm and 280 nm, between 180 nm and 260 nm, between 180 nm and 240 nm, or between 180 nm and 220 nm.
   10. The nanorobotic system of any one of paragraphs 1-9, wherein the system is configured to quantify analyte concentrations between 0.1 pg/ml and 100 pg/ml, between 0.1 pg/ml and 90 pg/ml, between 0.1 pg/ml and 80 pg/ml, between 0.1 pg/ml and 70 pg/ml, between 0.1 pg/ml and 60 pg/ml, between 0.1 and 50 pg/ml, between 0.1 pg/ml, and 40 pg/ml, between 0.1 pg/ml and 30 pg/ml, between 0.1 pg/ml and 20 pg/ml, between 0.1 pg/ml and 10 pg/ml, or between 0.1 pg/ml and 1 pg/ml.
   11. The nanorobotic system of any one of paragraphs 1-10, wherein the signal communication module contains one or more piezoelectric materials, circuits, or a combination thereof.
   12. The nanorobotic system of paragraph 11, wherein the one or more piezoelectric materials contain a quartz, a lead zirconate titanate, a barium titanate, a zinc oxide, an aluminum nitride, a polyvinylidene fluoride (PVDF), a lithium niobate, a gallium orthophosphate, a tourmaline, or a combination thereof.
   13. The nanorobotic system of paragraph 11 or 12, wherein the one or more piezoelectric materials contains a PVDF.
   14. The nanorobotic system of any one of paragraphs 11 to 13, wherein the AR system contains a scanning system (e.g., a microscopic scanning system), a real-time control module, and/or the AR user interface.
   15. The nanorobotic system of paragraph 14, wherein the real-time control module contains a first controller, a haptic device, a data acquisition board, a signal access module, and an augmented computer.
   16. The nanorobotic system of paragraph 14 or 15, wherein the scanning system contains a probe-laser system, a XYZ piezoelectric scanner, a conventional interface computer, a second controller, or a combination thereof, optionally wherein the first controller and the second controller are the same or different.
   17. The nanorobotic system of any one of paragraphs 14 to 16, wherein the scanning system contains an overhead microscope, a secondary inverted optical microscope, a CCD (charged couple device) camera, or combination thereof.
   18. The nanorobotic system of any one of paragraphs 1-17, wherein the analyte is a protein, optionally wherein the protein is a human chorionic gonadotropin (HCG).
   19. The nanorobotic system of any one of paragraphs 1-18, wherein the sample is a spent culture medium, optionally wherein the spent culture medium is from an embryo.
   20. The nanorobotic system of any one of paragraphs 1 to 19, containing a cantilever design, containing a 3-dimensional piezoelectric element attached to the cantilever for precise motion control of the functionalized probe.
   21. A method for detecting concentration levels of an analyte using the nanorobotic system of any one of paragraphs 1 to 20, the method involving measuring a signal indicating an interaction between the analyte on the platform and the functionalized probe.
   22. The method of paragraph 21, further involving:
generating a height and a rupture force of the analyte (e.g., protein) immobilized on the platform (e.g., scaffold disk) using atomic force microscopy (AFM), and/or
processing the height and the rupture force to plot the relative distributions of generated objects by height and adhesive force.
   23. The method of paragraph 21 or 22, further involving evaluating embryo quality in in vitro fertilization (IVF) treatments, by measuring β-HCG concentrations in spent culture medium (SCM) from embryo and/or categorizing the embryo into successful and unsuccessful pregnancy outcome groups based on their β-hCG levels.
   24. The method of any one of paragraphs 21 to 23, wherein adhesion forces exceeding 120 pN indicate the presence of the analyte engaging with the functionalized probe.
   25. The method of any one of paragraphs 21 to 24, wherein the nanorobotic system utilizes adaptive scanning patterns to detect new positions of the analyte during the protein measurement experimentation.

The methods and systems herein described are further illustrated in the following examples, which are provided by way of illustration and are not intended to be limiting. It will be appreciated that variations in proportions and alternatives in elements of the components shown will be apparent to those skilled in the art and are within the scope of disclosed forms. All parts or amounts, unless otherwise specified, are by weight.

### EXAMPLES

### Example 1: Nanorobotic System that Recognizes Proteins at Ultra-Low Concentrations

### Materials and Methods

The fabrication of the high aspect probe is described in FIGS. 3A and 3B. The initial phase of the study involved the precision crafting of a probe by employing a focused ion beam (FIB) milling process to refine a commercial cantilever ScanAsyst-Fluid (commercially available probe for imaging cells and related samples in fluid), which has a resonant frequency of 70 kHz and a force constant of 0.7 N/m. The FIB parameters were meticulously set: the ion beam current ranged from 10 to 50 pA, the ion beam dwell time was fixed at 1.0 s, and the ion beam accelerating voltage was maintained at 30 keV. Electron microscopy images of the FIB-fabricated dual probe were analyzed to ascertain the tip characteristics. The resulting probe featured a tip radius of approximately 50 nm and an inter-tip distance of about 200 nm. This tip configuration was achieved post-manufacturing, as confirmed by the image analysis.

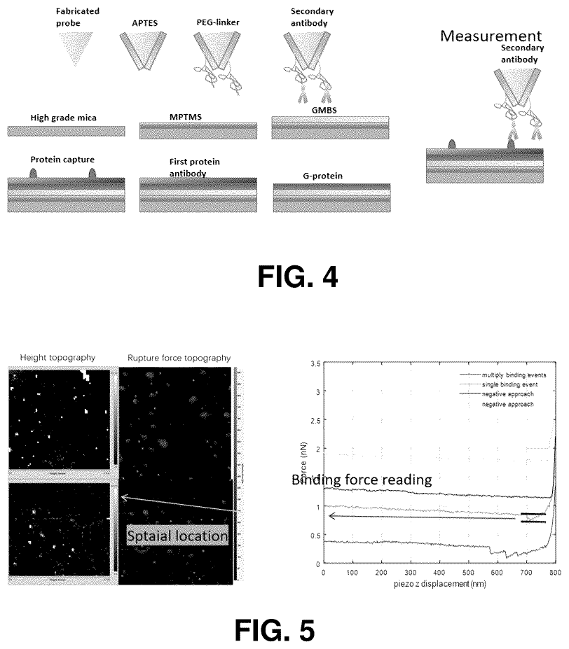

The detailed experimental protocol for performing the nanorobotic measurements on the targeted protein in the embryo is described below and in FIG. 4. When preparing the substrate G-protein-HCGAB, first, the glass slides are immersed in acetone for 10 min to remove organic impurities and then rinsed with DI water and ethanol. The slides are then dried under a stream of nitrogen. The glass slides are immersed in a 2% v/v solution of MPTMS in ethanol for 45 min at R.T. The slides are thoroughly rinsed with ethanol and dry under a stream of nitrogen. The slides are then immersed in 5 mM GMBS in DMSO for 30 min at R.T. The slides are thoroughly rinsed with DI water and dried under a stream of nitrogen. Protein G is dissolved in PBS (pH 7.4) to a concentration of 100 μg/mL. The slides are immersed in the Protein G solution (50 μL) and incubate for 45 min at R.T. The slides are then thoroughly rinsed with PBS to remove unbound Protein G. The first HCGAB is dissolved to the concentration of 100 μg/mL. The first HCGAB is then applied to the coated slides and incubated in a humid chamber for 60 min. The slides are rinsed with PBS to remove unbound antibodies, immersed in a solution of 1% BSA for 30 min to block at room temperature (R.T.), and finally rinsed with PBS.

For preparation of the probe through secondary hCGab functionalization, the probe is first cleaned with acetone for 10 min. The slides are rinsed with DI water, then ethanol, and dried under a stream of nitrogen. The probes are immersed in 10% v/v solution of APTES in acetone for 60 min at R.T, then rinsed thoroughly with DI water and dried under a stream of nitrogen. The probes are then immersed in the PEG-linker (O,O′-Bis[2-(N-Succinimidyl-succinylamino) ethyl]polyethylene glycol) (100 μg/mL) (50 μL) diluted with PBS and incubate for 45 min at R.T. The probes are rinsed gently with DI water. The secondary hCGab is incubated to the probes in a humid chamber for 45 min. The probes are gently rinsed with DI water, and then immersed in a solution of 1% BSA for 30 min to block at R.T. The probes are rinsed with DI water and stored in the PBS solution.

For the construction of a standard assay, the pure β-HCG is diluted to the concentration of 0-100 μg/ml (0; 0.5; 1; 2; 5; 10; 20; 50; 100). The sensor disk is incubated with β-HCG (10 μL) in humid chamber for 45 min at R.T, and the substrate is immersed with the PBS for measurement.

Evaluation of HCG in SCM starts with incubation of the sensor disk with SCM medium (10 μL) for 45 min at R.T, and then the disk is rinsed with DI water. The substrate is then immersed with the PBS for nanorobotic measurement.

The acquisition of binding event images or the protein distribution images are described in FIG. 5. The protein binding events were described by the rupture force value of the antibody and protein. The force-distance curves were obtained in the force curve version. The curves were recorded with a moderated applied force less than 5 nN, a ramp size of less than 2 μm and a ramp frequency of 1 hz. Each force-distance curve included the approach part and retract part, where the tips approached to and departed from the disk, respectively. For each measurement, a map of 64×64 curves was recorded in a 5 μm×5 μm area. The adhesive forces of rupture events were obtained from the retract part of the curves, which is equal to the force difference at the contact point and adhesive force peak location.

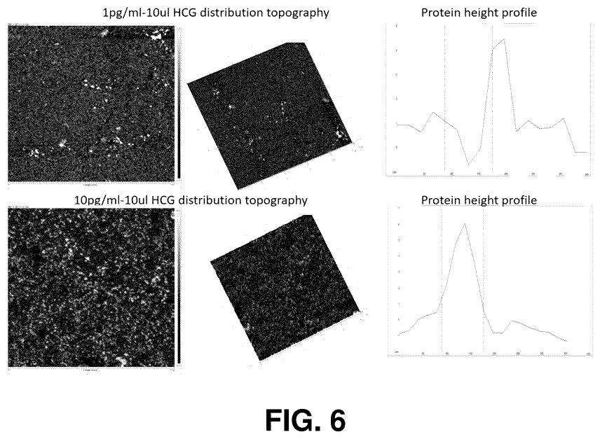

The preliminary measurement has shown the swift response of the HCG protein accumulation on the disk at low concentrations (less than 10 pg/ml for 10 μl). FIG. 6 shows the high-resolution topography of the HCG on the scaffold disk within 1 pg/ml and 10 pg/ml conditions. The captured protein increased dramatically by minorly increasing the dosage.

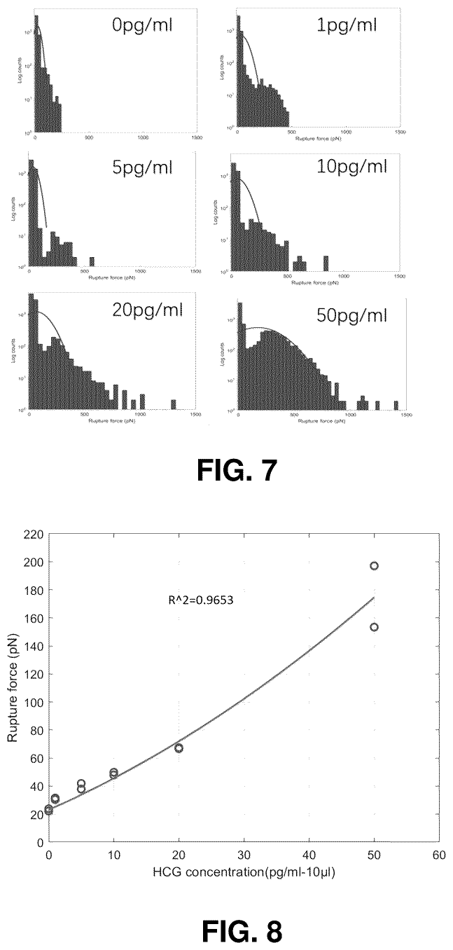

To establish the standard assay for the protein concentration calculation, the test has been set up to establish the relationship for the rupture events force-protein concentration and rupture force images-protein concentration. In this case, the pure HCG medium is used as a sample for the establishment of a standard assay and dataset for the neural network. Start from the control group (0 pg/ml) to 1 pg/ml; 5 pg/ml; 10 pg/ml; 20 pg/ml; 50 pg/ml; 100 pg/ml; 200 pg/ml. The mean value of the force among the test varies from 21.3 pN for the control to 287.1 pN for 200 pg/ml as shown in FIG. 7. After acquisition of the protein attachment data, the tentative regression curve was established by the regression method using the mean value and the pre-set protein concentration as shown in FIG. 8, to facilitate assessing establishment of the accuracy and efficiency of the regression curve.

In some instances, the signal communication module of the present system can include an augmented reality (AR) system. The AR system can involve a pair of 3D goggles for stereo visual feedback to mimic nanoscale operation with depth information This AR system can encompass three main components: a microscopic scanning system, a real-time control module which includes the controller, haptic device, signal access module and augmented computer, and the AR user interface.

The microscopic scanning system can include a probe-laser system, the XYZ piezoelectric scanner, conventional interface computer, and the controller, optionally including additional equipment such as an overhead microscope, a secondary inverted optical microscope, and a CCD (charged couple device) camera, providing optical microscopy's low-resolution videos of samples and probes. The real-time control module is fitted with a data acquisition board for data collection and a signal output to the signal access module. Data and control signals are operably linked between the user's computer and the conventional interface computer, optionally using the ethernet and UDP protocol. The AR platform updates scanning images in real time, using the spatial and force data gathered during nanomanipulation. A purpose-built signal-access module taps the scanner controller's outputs, and bespoke software processes the incoming data with dedicated algorithms.

### AI-Based Data Analytic System

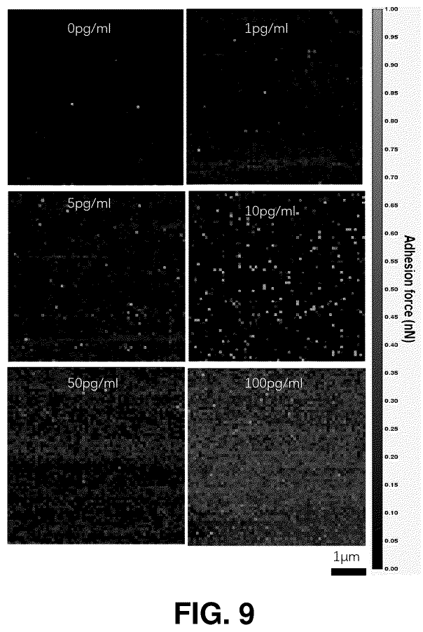

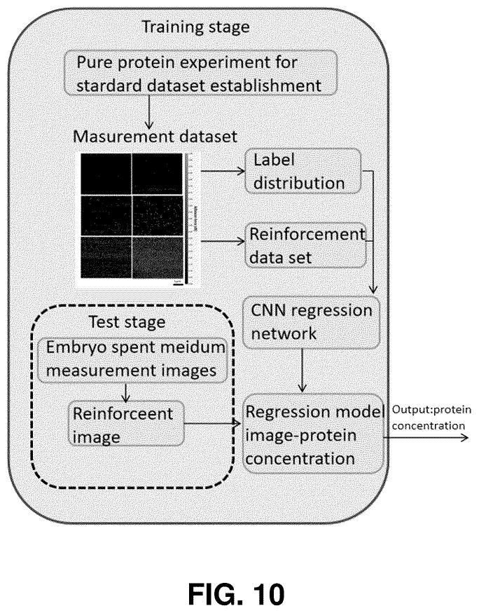

An AI-based data analytic system utilizing convolutional neural networks (CNNs) is used for interpreting protein-protein interaction images and converting them into accurate concentration readings. After high-resolution images of protein binding events are obtained, the images are post processed with the same standard to construct the training data with artificial labels as shown in FIG. 9. The images are then fed into a specifically tailored convolutional neural network to accurately estimate protein concentrations. To prepare these images for the CNN, they first undergo a series of preprocessing steps designed to enhance and standardize the features important for concentration estimation. These steps include rescaling the images to a uniform size suitable for the CNN, normalizing the pixel values to aid in the training process, and augmenting the image dataset with modified versions by redistribution the protein pixels among the images. FIG. 10 shows the flowchart of the architecture of the protein decision system.

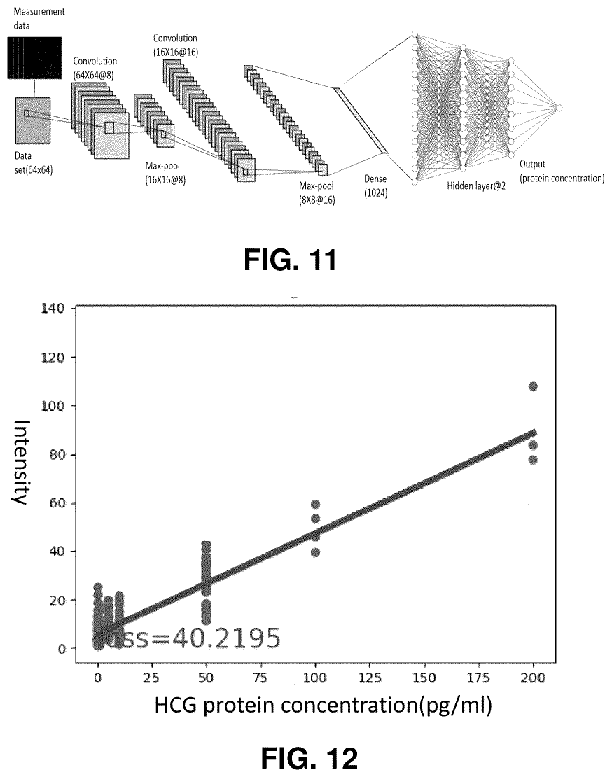

The deep learning network has been generated to solve a regression problem by using a straightforward model. The architecture of the CNN model used in this study is illustrated in FIG. 11. To generate singular floating-point numbers that denotes the estimated protein concentration in pg/ml, this model was modified to accommodate input images with a dimension and has convolutional layers that function as feature extractors equipped with a varying number of neurons and again using the ReLU activation function. Max pooling layers then reduce the spatial dimensions of the feature maps, which are subsequently flattened into a vector that feeds into dense layers. The dimension of the output image from convolution layers is flattened into a vector of size for forwarding to the next regression input layer. The regression layers consist of three fully-connected layers with 2048 hidden nodes. Since the output of this model represents the estimated protein concentration (pg/ml), the final output layer is a single neuron and a linear activation function for the regression task of estimating protein concentrations.

### Results

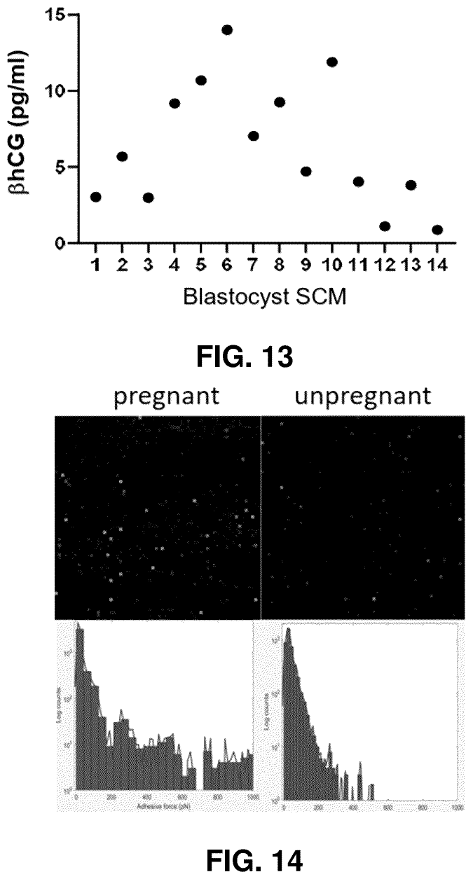

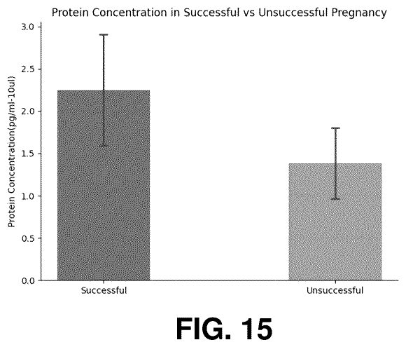

The training results with the training loss for evaluating the training results is shown in FIG. 12. The preliminary clinical test has also been applied to test the accessibility of the proposed analytical system. Immobilized anti-HCG antibodies were utilized to capture betaHCG present in the human spent culture medium (SCM). To quantify betaHCG molecules within a specified area, adhesion forces exceeding 120 pN indicate the presence of a betaHCG molecule engaging with the functionalized probe. The betaHCG concentration within a 10 μl volume of spent culture medium (SCM) was determined to range from 0.87 pg/ml to 14.00 pg/ml as shown in FIG. 13. Disclosed herein is a linear correlation for betaHCG concentrations ranging from 1 to 50 pg/ml, correlating with the number of adhesion sites, whereas the ELISA absorbance did not demonstrate this relationship. Further analysis involved assessing betaHCG levels in women undergoing IVF treatments, categorizing them into two distinct groups based on their pregnancy outcomes: successful and unsuccessful as shown in FIG. 14. Nanorobot detection indicated a progressive increase in betaHCG for both groups. Specifically, PHCG concentrations were analyzed in SCM from individual embryos and discovered that levels in the successfully pregnant group varied from 0.39 pg/ml to 4.809 pg/ml, while in the unsuccessful group, they ranged from 0.309 pg/ml to 2.63 pg/ml. FIG. 15 shows the successful group has the betaHCG level of 2,25±0.66 pg/ml and 1.38±0.42 pg/ml, establishing that the disclosed system can be used as an evaluation approach to access the grade of embryo. The quality of an embryo can be evaluated with the disclosed nanorobotic system for in-vitro fertilization (IVF) treatments, by measuring β-HCG concentrations in spent culture medium (SCM) from the embryos and categorizing the embryos into successful and unsuccessful pregnancy outcome groups based on their β-HCG levels.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

Those skilled in the art will recognize, or be able to ascertain using no more than routine experimentation, many equivalents to the specific embodiments of the invention described herein. Such equivalents are intended to be encompassed by the following claims.

We claim:

1. A nanorobotic system for detecting a concentration of an analyte in a sample, the system comprising:
a measurement module, comprising a functionalized probe and a platform (e.g. a scaffold disk),
a signal communication module comprising a material capable of generating and/or transmitting one or more signals optionally selected from electric signals, electrochemical signals, optical signals, mechanical signals (e.g., originating from pressure, tensile/compressive forces, accelerations, vibrations, strain/deformation, displacement, and/or shearing), acoustic signals, temperature signals, digital signals, and
a sample-tip positioning module (e.g., precise sample tip position module),
wherein the system is operably linked to an artificial intelligence (AI)-based data analytic system configured to interpret data comprising analyte-functionalized probe interaction images and/or convert the data into a concentration reading,
optionally wherein the AI-based data analytic system utilizes a machine learning AI platform, deep learning AI platform (e.g., neural networks such as convolutional neural networks (CNNs)), or a combination thereof, for interpreting the data (e.g., analyte-functionalized probe interaction images, such as protein-protein interaction images) and/or converting the data into concentration readings.

2. The nanorobotic system of claim 1, wherein the nanorobotic system is configured to implement a semantic compressive feedback control mechanism to interpret the data optionally by streamlining an image recognition process and/or reducing computational load.

3. The nanorobotic system of claim 1, wherein the functionalized probe comprises metals, metal alloys, carbon-based materials, polymers, magnetic materials, semiconductor materials, nucleic acid origamis (e.g., DNA origamis), or combinations thereof.

4. The nanorobotic system of claim 1, wherein the functionalized probe comprises alkoxysilane molecules on an external surface, wherein the alkoxysilane molecules are (3-aminopropyl)triethoxysilane (APTES), (3-mercaptopropyl)trimethoxysilane (MPTMS), (3-glycidyloxypropyl)trimethoxysilane (GPTMS), (3-chloropropyl)triethoxysilane (CPTES), (3-methacryloxypropyl) trimethoxysilane (MAPTMS), (3-triethoxysilylpropyl)succinic anhydride (TESPSA), trimethoxypropylsilane (TMPS), octadecyltrichlorosilane (OTS), hexamethyldisilazane (HMDS), tetraethoxysilane (TEOS), or a combination thereof.

5. The nanorobotic system of claim 1, wherein the functionalized probe comprises a linker, wherein the linker comprises a polyethylene glycol (PEG) linker, alkyl chain, disulfide bond, amide linker, ester linker, maleimide linker, hydrazone linker, thioether linker, carbamate linker, or a linker derived from click chemistry (e.g., triazole).

6. The nanorobotic system of claim 1, wherein the functionalized probe comprises molecules and/or chemical groups that bind to the analyte immobilized on the platform.

7. The nanorobotic system of claim 1, wherein the platform (e.g., scaffold disk) comprises a mica material (e.g., an AI grade muscovite mica material layer).

8. The nanorobotic system of claim 1, wherein the functionalized probe has:
(i) a tip radius between 10 nm and 100 nm, between 10 nm and 90 nm, between 10 nm and 80 nm, between 10 nm and 70 nm, between 10 nm and 60 nm, between 20 nm and 60 nm between 30 nm and 60 nm, or between 40 nm and 60 nm, and/or
(ii) a length between 50-300 μm, 50-250 μm, 50-200 μm, 50-150 μm, 100-150 μm, 100-200 μm, 100-250 μm, or 100-300 μm.

9. The nanorobotic system of claim 1, wherein the functionalized probe has an inter-tip distance between 100 nm and 300 nm, between 120 nm and 300 nm, between 140 nm and 300 nm, between 160 nm and 300 nm, between 180 nm and 300 nm, between 180 nm and 280 nm, between 180 nm and 260 nm, between 180 nm and 240 nm, or between 180 nm and 220 nm.

10. The nanorobotic system of claim 1, wherein the system is configured to quantify analyte concentrations between 0.1 pg/ml and 100 pg/ml, between 0.1 pg/ml and 90 pg/ml, between 0.1 pg/ml and 80 pg/ml, between 0.1 pg/ml and 70 pg/ml, between 0.1 pg/ml and 60 pg/ml, between 0.1 and 50 pg/ml, between 0.1 pg/ml, and 40 pg/ml, between 0.1 pg/ml and 30 pg/ml, between 0.1 pg/ml and 20 pg/ml, between 0.1 pg/ml and 10 pg/ml, or between 0.1 pg/ml and 1 pg/ml.

11. The nanorobotic system of claim 1, wherein the signal communication module comprises one or more piezoelectric materials, connector boards, and/or center mother boards.

12. The nanorobotic system of claim 11, wherein (i) the one or more piezoelectric materials comprise a quartz, a lead zirconate titanate, a barium titanate, a zinc oxide, an aluminum nitride, a polyvinylidene fluoride (PVDF), a lithium niobate, a gallium orthophosphate, or a tourmaline, or (ii) the one or more piezoelectric materials comprise a PVDF.

13. The nanorobotic system of claim 1, wherein the analyte is a protein, optionally wherein the protein is a Human chorionic gonadotropin (HCG).

14. The nanorobotic system of claim 1, wherein the sample is a spent culture medium, optionally wherein the spent culture medium is from an embryo.

15. The nanorobotic system of claim 1, comprising a cantilever design, comprising a 3-dimensional piezoelectric element attached to the cantilever for precise motion control of the functionalized probe.

16. A method for detecting concentration levels of an analyte using the nanorobotic system of claim 1, comprising measuring a signal indicating an interaction between the analyte on the platform and the functionalized probe.

17. The method of claim 16, further comprising:
generating a height and a rupture force of the analyte (e.g., protein) immobilized on the platform (e.g., scaffold disk) using atomic force microscopy (AFM), and/or
processing the height and the rupture force to plot the relative distributions of generated objects by height and adhesive force.

18. The method of claim 16, further comprising evaluating embryo quality in in vitro fertilization (IVF) treatments, by measuring β-HCG concentrations in spent culture medium (SCM) from embryo and/or categorizing the embryo into successful and unsuccessful pregnancy outcome groups based on their β-hCG levels.

19. The method of claim 16, wherein adhesion forces exceeding 120 pN indicate the presence of the analyte engaging with the functionalized probe.

20. The method of claim 16, wherein the nanorobotic system utilizes adaptive scanning patterns to detect new positions of the analyte during the protein measurement experimentation.
